(19) United States
(12) Patent Application Publication
(10) Pub. No.: US 2007/0073220 A1
(43) Pub. Date: Mar. 29, 2007

(54) Inoculation device

(76) Inventor: Philip Bunce, Portland (AU)

Correspondence Address: MERCHANT & GOULD PC, P.O. BOX 2903, MINNEAPOLIS, MN, 55402-0903, US

(21) Appl. No.: 10/574,822

(22) PCT Filed: Oct. 6, 2004
(86) PCT No.: PCT/AU04/01362

§ 371 Date: Apr. 6, 2006

(51) Int. Cl. A61M 5/00

(52) U.S. Cl. 604/110

(57) ABSTRACT

An inoculation device comprising a body having a fixed portion and a moveable portion, the fixed portion having a front face the moveable portion supported from the fixed portion rearward of the front face, a needle supported from the movable portion to be moveable on relative longitudinal movement of the movable portion with respect to the fixed portion, towards and away from the front face whereby free end of the needle is moveable between a retracted position at which the free end of the needle lies to the rear of the front face and an extended position at which the free end of the needle extends in front of the front face, said movable portion being biased to the retracted position, an indexing means provided between the moveable portion and the fixed portion to cause lateral movement of the needle relative to the longitudinal movement in association with the longitudinal movement of the needle

### FIELD OF THE INVENTION

This invention relates to an inoculation means.

Throughout the specification, unless the context requires otherwise, the word “comprise” or variations such as “comprises” or “comprising”, will be understood to imply the inclusion of a stated integer or group of integers but not the exclusion of any other integer or group of integers.

### BACKGROUND OF THE INVENTION

This invention relates to a means to facilitate inoculation of patients which requires the creation of a number of punctures in the skin of a patient. An example of the application of the invention relates to inoculation against small pox by utilisation of a suitable vaccine. It is a characteristic of such inoculation that rather than inject the medium into the muscle tissue of the body it is necessary to introduce the medium into the skin or epidermis of the patient at a number of locations.

### DISCLOSURE OF THE INVENTION

According to one aspect the invention resides in an inoculation device comprising a body having a fixed portion and a moveable portion, said fixed portion having a front face, the moveable portion supported from the fixed portion rearward of the front face, a needle supported from the movable portion to be moveable on relative longitudinal movement of the movable portion with respect to the fixed portion, towards and away from the front face whereby free end of the needle is moveable between a retracted position at which the free end of the needle lies to the rear of the front face and an extended position at which the free end of the needle extends in front of the front face, said movable portion being biased to the retracted position, an indexing means provided between the moveable portion and the fixed portion to cause lateral movement of the needle relative to the longitudinal movement in association with the longitudinal movement of the needle

According to a preferred feature of the invention the front face is closed and is provided with an aperture which defines a path for the lateral movement of the needle and through which the needle extends when in its extended position.

According to a preferred feature of the invention the indexing means causes the needle to move to a final position located beyond the path defined by the aperture on completion of passage along the path.

According to a preferred feature of the invention the needle occupies an initial position prior to undergoing any longitudinal movement at which the needle is retracted and the needle is out of longitudinal alignment with the path and on the initial movement of the needle it moves into longitudinal alignment with the path.

According to another aspect the invention resides in an inoculation device comprising a body having a fixed portion and a moveable portion, said fixed portion comprising a substantially cylindrical body having a front face, the moveable portion being slidably and rotatably within the body, a needle supported from the movable portion to be moveable on relative longitudinal movement of the movable portion with respect to the fixed portion, towards and away from the front face whereby free end of the needle is moveable between a retracted position at which the free end of the needle lies rearward of the front face and an extended position at which the free end of the needle extends forward of the front face, said movable portion being biased to the retracted position, the needle being supported such that its central axis is offset from the axis of rotation of the movable portion within the fixed portion an indexing means provided between the moveable portion and the fixed portion to cause lateral movement of the needle relative to the longitudinal movement about the axis of rotation, in association with the longitudinal movement of the needle

According to a preferred feature of the invention the movable portion is capable of rotational movement in one direction about the axis of rotation.

According to a preferred feature of the invention the front face is closed and is provided with an at least partially annular aperture which defines a path for the lateral movement of the needle and through which the needle extends when in its extended position. According to one embodiment the aperture is part annular. According to a preferred feature of the invention the indexing means causes the needle to move to a final position located beyond the path defined by the aperture on completion of passage along the path. According to a preferred feature of the invention the needle occupies an initial position prior to undergoing any longitudinal movement at which the needle is retracted and the needle is out of longitudinal alignment with the path and on the initial movement of the needle it moves into longitudinal alignment with the path.

According to a preferred feature of both aspects of the invention the indexing means is adapted to cause the lateral movement whilst the needle is undergoing its longitudinal movement. According to a preferred feature the indexing means is adapted to cause the lateral movement during the movement of the needle from its retracted position to its extended position. According to an alternative preferred feature the indexing means is adapted to cause the lateral movement during the movement of the needle from its extended position to its retracted position. According to an alternative preferred feature the indexing means is adapted to cause the lateral movement during the movement of the needle from its retracted position to its extended position and from its extended position to its retracted position.

According to a preferred feature of both aspects of the invention the front face is open.

According to a preferred feature of both aspects of the invention the lateral movement is effected prior to the needle undergoing its longitudinal movement.

According to a preferred feature of both aspects of the invention the lateral movement is effected subsequent to the needle undergoing its longitudinal movement.

The invention will be more fully understood in the light of the following description of several specific embodiments.

### BRIEF DESCRIPTION OF THE DRAWINGS

The description is made with reference to the accompanying drawings of which:

FIG. 1 is an isometric front view of inoculation device according to the first embodiment;

FIG. 2 is a sectional side elevation of the first embodiment;

FIG. 3 is a rear exploded isometric view of the first embodiment;

FIG. 4 is a front exploded isometric view of the first embodiment;

FIG. 5 is a sectional side elevation of the base of the first embodiment;

FIG. 6 is a side elevation of the moveable portion of the first embodiment;

FIG. 7 is a front elevation of the moveable portion of the first embodiment;

FIG. 8 is a sectional side elevation of the moveable portion of the first embodiment;

FIG. 9 is a rear isometric view of the rotary member of the first embodiment;

FIG. 10 is a front isometric view of the rotary member of the first embodiment;

FIG. 11 is a side elevation of the closure of the first embodiment; and

FIG. 12 is a sectional side elevation of the closure of the first embodiment.

### DETAILED DESCRIPTION OF SPECIFIC EMBODIMENT

The first embodiment which is shown in the drawings relates to an inoculation device which can be utilised in inoculating patient with a vaccine where the process requires creating a number of punctures in the skin to enable the introduction of the vaccine into the body.

The first embodiment comprises a base 11 which supports a moveable member 13 which in turn supports a needle 15. The base 11 is formed as a cylindrical member having an open front face which is defined by an annular flange 17. The open front face of the base 11 supports a closure 19 which is fixedly supported from the base 11. The rear most end of the base 11 is formed with a waisted portion 23. The interior of the base 11 adjacent the waisted portion 23 is formed with a first tooth shaped formation 25 in which the teeth 25 are directed axially towards the front face of the base.

The closure member 19 is fixedly received in the open end of the front face of the base 11 and comprises a circular panel 26 which closes the open front face of the base 11. When the closure member 19 is located in the base 11 the outermost face of the circular panel 26 is offset rearwardly from the open end of the base 11 in order that when the base is in engagement with a substantially flat surface the outermost face of the circular panel is spaced away from the surface.

The closure member 19 further comprises a skirt 27 located at the rear face of the front panel 26 where the outer diameter of the skirt is complementary to the inner diameter of the base 11. The skirt 27 is formed around its most edge with a second tooth shaped formation 29 which is of corresponding form to the first tooth shaped formation 29. The closure member is supported within the base such that the peaks of the first tooth shaped formation 25 are slightly angularly offset from the peaks of the second tooth shaped formation 29.

In addition the internal radial face of the skirt 27 of the closure member at a position adjacent the front panel 26 is formed with a set of ratchet like teeth 31 (see FIG. 12) which extend for a portion of the inner circumference of the skirt.

The front panel 26 is formed with aperture 21 which is of a part annular configuration which is concentric with the centre of the panel.

The moveable portion 13 comprises a cylindrical body which is slidably received within the waisted end 23 of the base 11 to be axially slidable within the base 11. The innermost end of the moveable member 13 is provided around its outer perimeter with a plurality of radial protrusions 33 which are intended to be engaged by the tooth shaped formations 25 and 29 of the base 11 and closure 19 respectively on relative axial displacement of the moveable member 23 within the base 11.

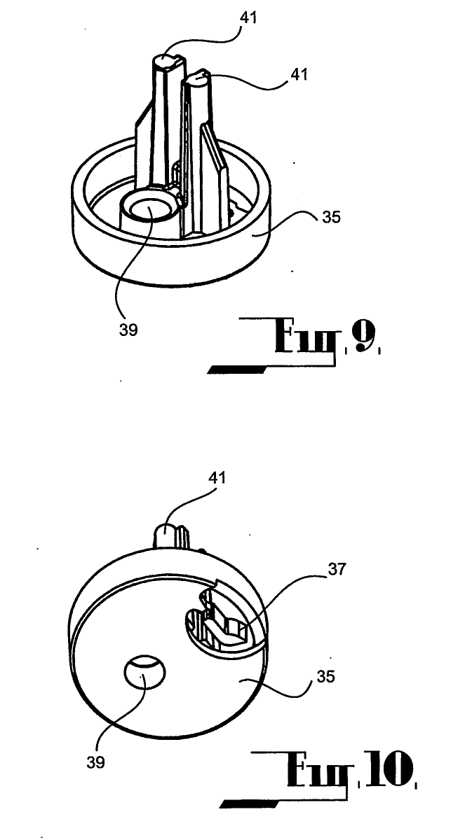

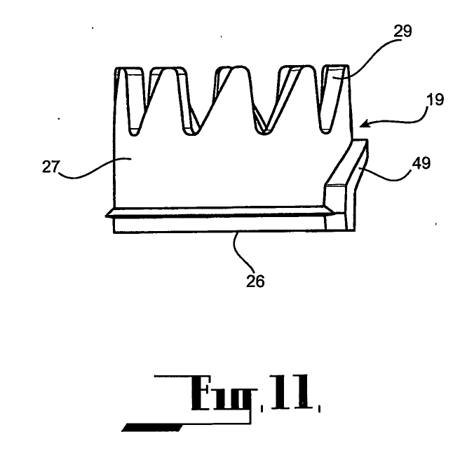

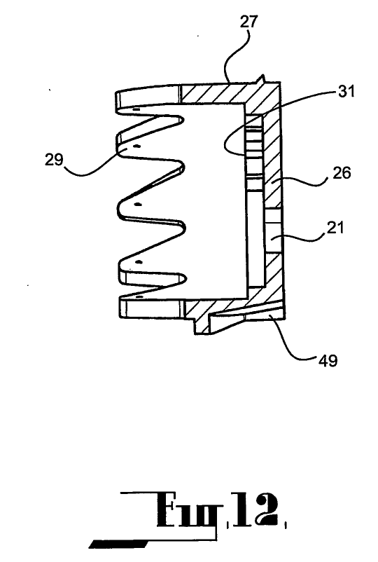

In addition the base portion 11 supports a rotary member 35 which is rotatably received within the skirt 27 of the closure 19 between the inner face of the panel 26 and the innermost end of the moveable member 13. The rotary member is provided with a radially directed pawl 37 having an outer edge which extends beyond the perimeter of the rotary member 35 such that it will bear upon the inner radial face of the skirt 27 to be engageable with the ratchet teeth 31 provided on the inner face. The interaction of the pawl 37 with the ratchet teeth 31 permits rotation of the rotary member 35 in one direction but not in the other. In addition the rotary member 35 is provided with a circular aperture 39 which is off centre and is intended to align with the aperture 21 of the closure member 19. The inner face of the rotary member 35 is provided with a pair of axially directed post members 41 which are spaced from each other (see FIGS. 9 and 10).

The interior of the moveable member 13 is provided with an axial spigot 43 which is provided with a bore which receives and fixedly supports the needle 15. The spigot is off-centre relative to the moveable member and consequently the needle is supported such that it is radially off-centre and the degree of radial offset corresponds to the radius of the part annular aperture 21 in the closure member 19. The spigot 43 is provided with an axial flange 45 which is slidably received between the post members 41 and the spacing between the post members 41 corresponds to the thickness of the axial flange 45.

A spring 47 is located within the moveable member to be located between the moveable member inner axial face of the moveable member 13 and the inner axial face of rotary member 35 and serves to bias the moveable member 13 axially outwardly from the base 11 and force the rotary member into face to face engagement with the inner-most face of the front panel 26 of the closure member 19.

The interrelationship between the moveable member 13 and the rotary member 35 is such that whilst the moveable member 13 is moveable axially within the rotary member 35 the two components are fixed rotationally and consequently rotation of the moveable member 13 results in rotation of the rotary member 35 and the direction of rotation of the movable member 13 and the rotary member 35 is controlled by the interaction of the pawl 37 of the rotary member with the ratchet teeth 31 of the closure.

In addition the interaction of the radial protrusions 33 of the movable member with the tooth formations 25 and 29 are such that on the moveable member 13 being moved inwardly the protrusion 33 will move from the first tooth shaped formation 25 into the second tooth face formation 29 and will be displaced angularly as a result of that interengagement. On the subsequent return of the moveable member 13 to its outer position with respect to the base 11 under the influence of the spring 47 the movement of the protrusion 33 from the second tooth shaped formation 29 to the first tooth shaped formation 25 will cause angular displacement of the moveable member with respect to the base 11. The interengagement of the pawl 37 with the ratchet teeth 31 will ensure that the angular movement of the moveable member 13 with respect to the base 11 is in the same direction.

The off centre positioning of the needle 15 causes the needle to move in a path around the centre of the closure as the movable member is indexed as a result of the interaction of the movable member with the toothed formations. At least a portion of that path corresponds with the part annular aperture 21. When the moveable member 13 is at its outermost at rest position with respect to the base 11 the free end of the needle 15 is located within the base 11 and rearward of the front face. On movement of the moveable member 13 into the base 11 and when the needle is aligned with the aperture the free end of the needle 15 will be caused to move through the aperture 21 such that its free end lies in front of the front face of the base 11.

In use it is intended that initially, the needle 15 will be located within the base to be aligned with one end of the part annular aperture 21 and with repeated depressions of the moveable member 13 with respect to the base 11 the needle will with each depression be caused to Move axially through the part annular aperture and beyond the outermost face of the front panel 26 of the closure member 19 will be indexed along the length of the part annular aperture 21. On the needle reaching the other end of the part annular aperture 21 it will move to a position lying inward of the panel and out of alignment with the part annular aperture 21 of the cover panel 26 of the closure member 19. When at this position the movable member will be prevented from undergoing sufficient axial movement within the base to be able to be indexed any further with any further depressions of the moveable member 13 with respect to the base 11 as a result of the outer end of the needle engaging with the innermost face of the panel 26 at which point in time the inoculation device will have reached the end of its usefulness and can be safely disposed.

In addition the outer cylindrical wall of the base in the region of the front face is formed with a cut away portion 47 and the corresponding portion of the skirt of the closure 19 is formed with a rib 49 of complementary configuration to the cutaway portion. The rib 49 co-operates with the cutaway portion of the base to prevent relative rotational movement therebetween. In addition the cut away portion provides a visual and tactile indicator to an operator using the device of the manner in which the device is to be used.

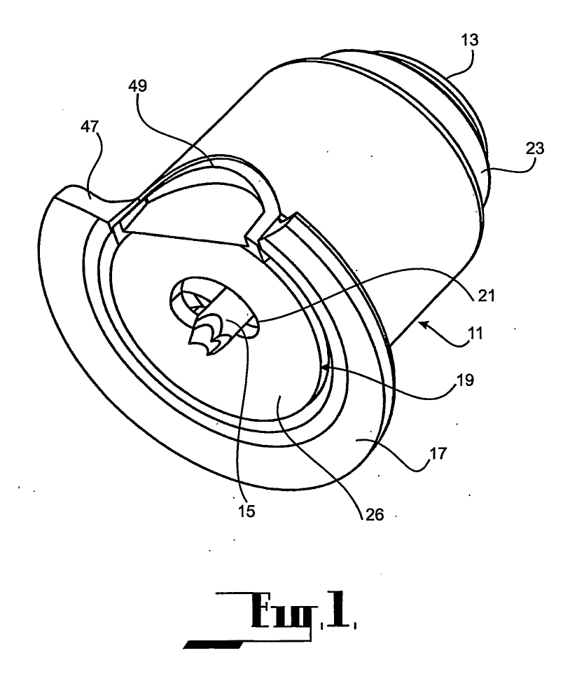

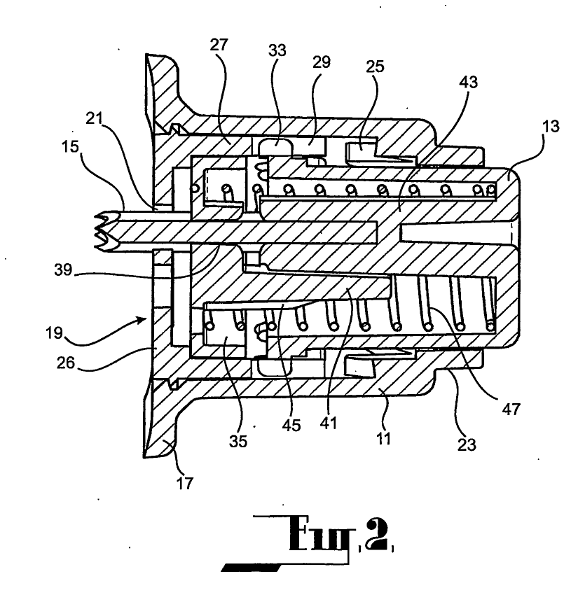

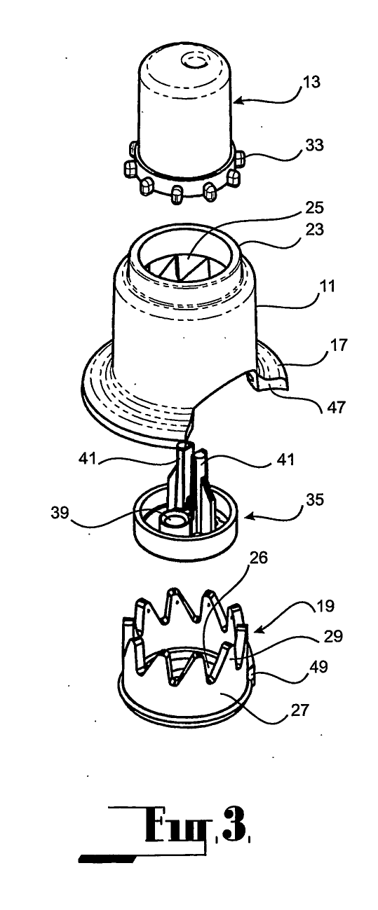

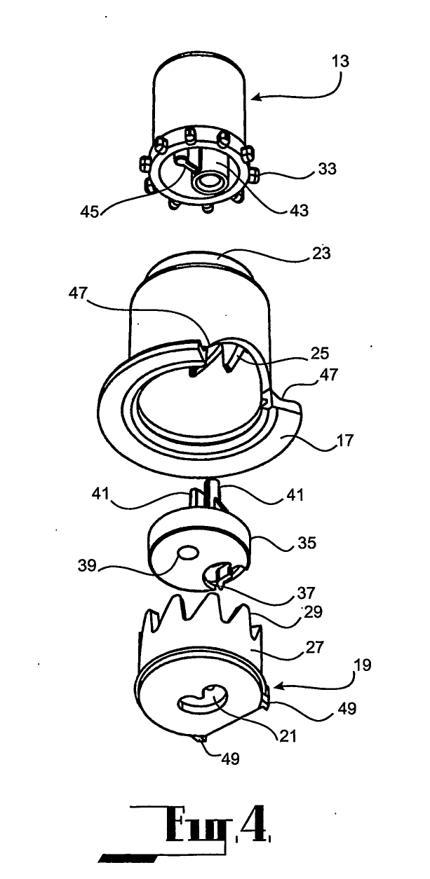

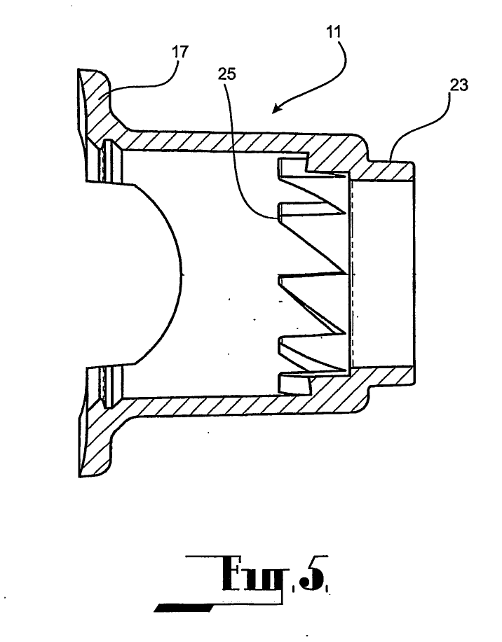

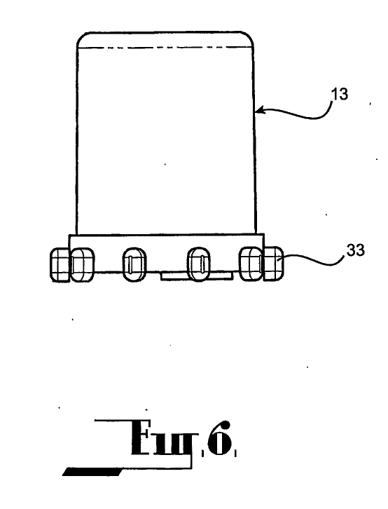

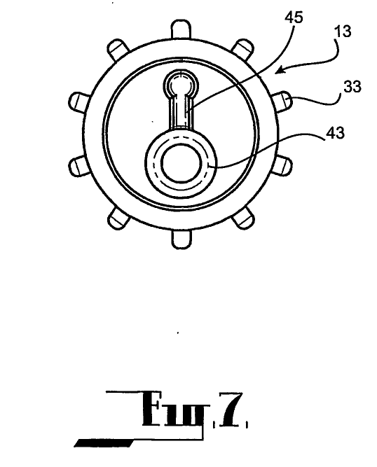

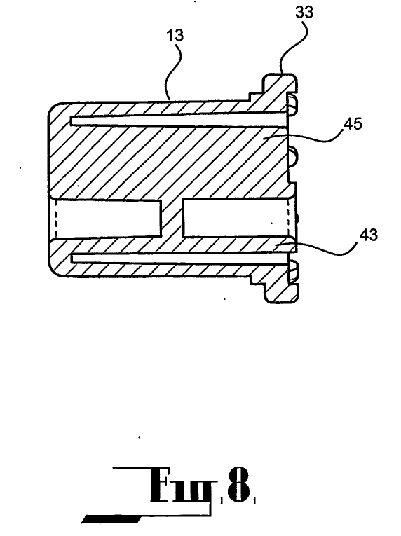

The free end of the needle 15 is provided with a number of points 49 such that with each excursion of the needle from the retracted position to the extended position as shown at FIGS. 1 and 2 the device will form a plurality of punctures in the skin of the patient. The degree of axial movement of the needle is such that when the needle is at its outermost position the points 49 individually pierce the skin to provide a circular array of punctures in the skin.

The effect of the inoculation device according to the first embodiment is that it is capable of repeatedly producing a number of circular arrays of punctures in a particular zone on the skin of a patient in order to effect adequate inoculation of a vaccine. Once the inoculation process has been completed the needle is safely accommodated within the device and the device cannot be used again and therefore can be safely discarded.

According to other embodiments of the invention the indexing means will cause the lateral indexing of the needle prior to the needle commencing its longitudinal movement, subsequent to the needle completing its longitudinal movement, on the needle moving towards its extended position or on the needle moving towards its retracted position.

It should be appreciated that the scope of the invention need not be limited to the particular scope of the embodiments described above.

1. An inoculation device comprising a body having a fixed portion and a moveable portion, said fixed portion having a front face, the moveable portion supported from the fixed portion rearward of the front face, a needle supported from the movable portion to be moveable longitudinally on relative movement of the movable portion with respect to the fixed portion, towards and away from the front face whereby the free end of the needle is caused to undergo a longitudinal movement between a retracted position at which the free end of the needle lies to the rear of the front face and an extended position at which the free end of the needle extends in front of the front face, said movable portion being biased to the retracted position, an indexing means provided between the moveable portion and the fixed portion to cause an indexing lateral movement of the needle in a direction transverse to the longitudinal movement through a plurality of stations in association with the longitudinal movement of the needle wherein on each longitudinal movement the needle is located at a separate station.

2. An inoculation device as claimed at claim 1 wherein the indexing means is adapted to cause the lateral movement during the movement of the needle is undergoing its longitudinal movement.

3. An inoculation means as claimed at claim 2 wherein the indexing means is adapted to cause the lateral movement during the movement of the needle from its retracted position to its extended position.

4. An inoculation means as claimed at claim 2 wherein the indexing means is adapted to cause the lateral movement during the movement of the needle from its extended position to its retracted position.

5. An inoculation means as claimed at claim 2 wherein the indexing means is adapted to cause the lateral movement during the movement of the needle from its retracted position to its extended position and from its extended position to its retracted position.

6. An inoculation device as claimed in claim 1 wherein the indexing means is adapted to cause the lateral movement during the movement of the needle prior to the needle undergoing its longitudinal movement.

7. An inoculation device as claimed in claim 1 wherein the indexing means is adapted to cause the lateral movement during the movement of the needle subsequent to the needle undergoing its longitudinal movement.

8. An inoculation means as claimed in claim 1 wherein the front face is open.

9. An inoculation means as claimed in claim 1 wherein the front face is closed and is provided with an aperture which defines a path for the lateral movement of the needle and through which the needle extends when in its extended position, said stations being located along said path.

10. An inoculation means as claimed at claim 9 wherein the indexing means causes the needle to move to a final station located beyond the path on completion of passage of the needle along the path.

11. An inoculation means as claimed in claim 9 wherein the needle occupies an initial position prior to undergoing any longitudinal movement at which the needle is retracted and the needle is out of longitudinal alignment with the path and on the initial movement of the needle it moves to an initial station and into longitudinal alignment with the path.

12. An inoculation device comprising a body having a fixed portion and a moveable portion, said fixed portion comprising a substantially cylindrical body having a front face, the moveable portion being slidably and rotatably within the body, a needle supported from the movable portion to be moveable longitudinally on relative movement of the movable portion with respect to the fixed portion, towards and away from the front face whereby the free end of the needle is caused to undergo a longitudinal movement between a retracted position at which the free end of the needle lies rearward of the front face and an extended position at which the free end of the needle extends forward of the front face, said movable portion being biased to the retracted position, the needle being supported such that its central axis is offset from the axis of rotation of the movable portion within the fixed portion an indexing means provided between the moveable portion and the fixed portion to cause an indexing lateral movement of the needle in a direction transverse to the longitudinal movement about the axis of rotation, in association with the longitudinal movement of the needle wherein on each longitudinal movement the needle is located at a separate station.

13. An inoculation means as claimed claim 12 wherein the movable portion is capable of rotational movement in one direction about the axis of rotation.

14. An inoculation means as claimed in claim 12 wherein the front face is open.

15. An inoculation means as claimed in claim 12 wherein the front face is closed and is provided with a an at least partially annular aperture which defines a path for the lateral movement of the needle and through which the needle extends when in its extended position, said stations being located along said path.

16. An inoculation means as claimed claim 15 wherein the aperture is part annular.

17. An inoculation means as claimed at claim 16 wherein the indexing means causes the needle to move to a final station is located beyond the path on completion of passage of the needle along the path.

18. An inoculation means as claimed in claim 16 wherein the needle occupies an initial position prior to undergoing any longitudinal movement at which the needle is retracted and the needle is out of longitudinal alignment with the path and on the initial movement of the needle it moves to an initial station and into longitudinal alignment with the path.

19. An inoculation device as claimed in claim 12 wherein the indexing means is adapted to cause the lateral movement whilst the needle is undergoing its longitudinal movement.

20. An inoculation means as claimed in claim 12 wherein the indexing means is adapted to cause the lateral movement during the movement of the needle from its retracted position to its extended position.

21. An inoculation means as claimed in claim 12 wherein the indexing means is adapted to cause the lateral movement during the movement of the needle from its extended position to its retracted position.

22. An inoculation means as claimed in claim 12 wherein the indexing means is adapted to cause the lateral movement during the movement of the needle from its retracted position to its extended position and from its extended position to its retracted position.

23. An inoculation device as claimed in claim 12 wherein the indexing means is adapted to cause the lateral movement prior to the needle undergoing its longitudinal movement.

24. An inoculation device as claimed in claim 12 wherein the indexing means is adapted to cause the lateral movement subsequent to the needle undergoing its longitudinal movement.
